SEQUENCE LISTING (4 sequences)

SEQ ID NO: 1 - protein, 14 aa, Homo sapiens
VHLTPVEKSAVTAC

SEQ ID NO: 2 - protein, 147 aa, Homo sapiens [sequence omitted]

SEQ ID NO: 3 - protein, 142 aa, Homo sapiens
MVLSPADKTNVKAAWGKVGAHAGEYGAEALERMFLSFPTTKTYFPHFDLSHGSAQVKGHGKKVADALTNAVAHVDDMPNALSALSDLHAHKLRVDPVNFKLLSHCLLVTLAAHLPAEFTPAVHASLDKFLASVSTVLTSKYR

SEQ ID NO: 4 - protein, 147 aa, Homo sapiens
MVHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH

(19) United States
(12) Patent Application Publication
(10) Pub. No.: US 2019/0041407 A1
(43) Pub. Date: Feb. 7, 2019

(54) DEVICES, SYSTEMS AND METHODS FOR QUANTIFYING HEMOGLOBIN S CONCENTRATION

(71) Applicant: BioMedomics, Inc., Durham, NC (US)

(72) Inventors: XiaoXi Yang, Durham, NC (US); Jason S. Kim, Raleigh, NC (US)

(21) Appl. No.: 16/076,489

(22) PCT Filed: Feb. 9, 2017
(86) PCT No.: PCT/US17/17210

§ 371 Date: Aug. 8, 2018

Related U.S. Application Data
(60) Provisional application No. 62/293,103, filed on Feb. 9, 2016.

(51) Int. Cl. G01N 33/72; C07K 14/805; G01N 33/531; C07K 16/18; G01N 33/543

(52) U.S. Cl. CPC G01N 33/726; C07K 14/805; G01N 33/54346; C07K 16/18; G01N 33/531

(57) ABSTRACT

Lateral flow immunoassay devices, systems and methods are provided for quantifying hemoglobin S in a sample. Point-of-care devices and methods for quickly and accurately monitoring a subject's level of hemoglobin S in a blood sample are provided.

### CROSS-REFERENCE TO RELATED APPLICATIONS

This application claims benefit of U.S. Provisional Patent Application Ser. No. 62/293,103, filed Feb. 9, 2016, herein incorporated by reference in its entirety.

### TECHNICAL FIELD

The present disclosure pertains to methods, systems and devices for measuring hemoglobin S (HbS) concentration in a sample. More specifically, the present disclosure relates to immunoassay approaches, including methods, systems and devices, to quantitatively measure hemoglobin S in whole blood specimens of sickle cell disease (SCD) patients who are undergoing therapy.

### BACKGROUND

Red blood cell (RBC) transfusion is the only proven treatment for both primary prevention in SCD patients and secondary stroke prevention in patients who have already suffered a stroke. While stroke will typically recur in about 60% of SCD patients [1], chronic RBC transfusions reduce the recurrence rate to only about 20% when HbS levels are maintained below 30% [2, 3]. Chronic RBC transfusions can be given as simple transfusions or as exchange transfusions using either automated apheresis machines or manual partial exchanges. These are usually performed every 3-4 weeks, and monitoring HbS concentration at each transfusion is essential to determine the appropriate number of RBC units needed to achieve HbS levels below 30% for effective reduction of stroke risk [4].

Hydroxyurea, the only FDA-approved drug for treatment of SCD, significantly decreases painful crises, acute chest syndrome, and hospitalizations [5] while improving patient survival [6]. Hydroxyurea works by increasing production of RBCs with higher levels of fetal hemoglobin (HbF), which interferes with the polymerization of HbS [7] to reduce sickling and vaso-occlusive events [8-10]. In 2010, 43% of sickle cell patients received hydroxyurea treatment [11], but recently released guidelines from the National Institutes of Health [12] have strong recommendations to expand hydroxyurea treatment. To effectively implement hydroxyurea treatment, a method for the quantitative measurement of HbF/HbS levels is required before and throughout treatment as a means for monitoring.

Current methods of measuring HbS levels (Hb electrophoresis or HPLC) require the use of complex, centralized laboratory equipment and highly-trained technicians. Results are not available to doctors and patients for several days after the test is ordered, which means the doctor must make treatment decisions without knowing the actual % HbS values for their patient. Therefore, SCD treatment is not based on the patient's real-time HbS level due to lag time of days for HbS test results from centralized laboratories. A point-of-care (POC) test that could be administered prior to and during treatment will ensure that patient receive the appropriate amount of RBCs/dose of Hydroxyurea.

Thus, there is an established need for inexpensive, easy-to-use, point-of-care test or assay to measure HbS levels in a patient sample. To address this unmet need for real-time HbS level monitoring, a rapid, cost effective immunoassay technique to quantify and/or monitor HbS concentration for patients undergoing SCD treatments could reduce devastating SCD complications and avoidable therapy-related adverse effects.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

In some embodiments a hemoglobin S (HbS) diagnostic or screening apparatus is provided, comprising a chromatography matrix, a detection antibody with binding affinity to hemoglobin (Hb), a detectable marker conjugated to the detection antibody, and one or more capture antibodies, wherein the capture antibodies have a binding affinity to HbS, Hb α chain, and/or Hb β chain, wherein the diagnostic or screening apparatus is configured as a lateral-flow immunoassay, and wherein the diagnostic or screening apparatus is configured to detect HbS in a sample from a subject. In some embodiments the detection and/or capture antibodies are monoclonal antibody (mAb) and/or polyclonal antibody (pAb). In some embodiments the detectable marker comprises a colored or fluorescent nanoparticle. In some embodiments the diagnostic or screening apparatus is configured to qualitatively and/or quantitatively detect and/or measure human HbS in human blood samples. In some embodiments the detection antibody is chemically conjugated to the detectable marker to form a permanent, irreversible antibody-marker complex. In some embodiments the sample from a subject is selected from the group consisting of a whole blood sample, a dried blood sample, a packed red cell sample, a whole blood sample or red blood sample from a blood bank, an isolated or purified human hemoglobin protein sample, and a freshly collected filter paper sample. In some embodiments the detectable marker is an enzyme label, fluorescent label, radiolabel, particulate label, colored latex particle, colored plastic particle, and a phosphor particle.

In some embodiments at least one of the capture antibodies has a binding affinity to HbS, optionally wherein the capture antibody comprises an antibody having an affinity to an amino acid sequence of SEQ ID NO. 1, wherein SEQ ID NO. 1 comprises an immunogenic amino acid sequence of HbS.

Also provided herein are point-of-care (POC) immuno assay devices comprising a chromatography maxtrix, a sample pad, a conjugate pad, a detection antibody with binding affinity to a hemoglobin (Hb), a detectable marker conjugated to the detection antibody, one or more capture antibodies, wherein the capture antibodies have a binding affinity to hemoglobin S (HbS), Hb α chain, and/or Hb β chain, and a wicking pad, wherein the device is configured to detect and/or quantify HbS in a sample from a subject at the point-of-care or in a field setting. In some embodiments the POC immuno assay is configured as a lateral-flow immunoassay. In some embodiments the immuno assay device is configured to assist in monitoring or guiding the transfusion therapy of sickle cell disease (SCD) patients. In some embodiments a housing can be configured to substantially enclose the chromatography maxtrix, sample pad, conjugate pad, detection antibody, capture antibodies and wicking pad, optionally in a disposable one-time use package.

In some embodiments the devices can further comprise a sample receiving area configured to receive a sample and a test window configured to allow observation of test results. In some embodiments the wicking pad is configured to cause a sample to migrate in a lateral direction from the sample pad to the wicking pad by capillary action. In some embodiments the one or more capture antibodies can be applied to the chromatography matrix and configured as a test line and control line, wherein both the test line and control line are visible in the test window. In some embodiments the detection and/or capture antibodies are monoclonal antibody (mAb) and/or polyclonal antibody (pAb). In some embodiments the capture antibodies further comprise an anti-HbS capture antibody and an anti-Hb capture antibody, wherein the anti-HbS capture antibody has an affinity to HbS, wherein the anti-Hb capture antibody has an affinity to Hb α chain and/or Hb β chain.

In some embodiments the detectable marker comprises a colored or fluorescent nanoparticle. In some embodiments the detection antibody is chemically conjugated to the detectable marker to form a permanent, irreversible antibody-marker complex. In some embodiments the detectable marker is an enzyme label, fluorescent label, radiolabel, particulate label, colored latex particle, colored plastic particle, and a phosphor particle. In some embodiments the detection antibody conjugated to the detectable marker is provided in a simplexed or multiplexed format to simultaneous detect, quantify and/or differentiate the presence or absence of HbS in a sample.

Also provided herein are methods for quantifying hemoglobin S (HbS) in a patient sample, comprising providing a sample from a patient, providing the diagnostic or screening apparatus, administering the sample to the diagnostic or screening test, and measuring an intensity of the detectable marker to quantify the HbS in the sample. In some embodiments the method is configured to provide a quantitative measure of HbS in the sample within about 30 minutes or less. In some embodiments the method is configured to assist in monitoring or guiding the transfusion therapy of sickle cell disease (SCD) patients. In some embodiments the method further comprises using an optical reader configured to detect a colorimetric intensity and/or fluorescence intensity.

Also provided herein are kits for measuring hemoglobin S in a sample, comprising a device or apparatus as disclosed herein, a sampler for collecting a blood sample, a buffer module containing a buffer, and instructions for performing measurement of hemoglobin S in the sample. In some embodiments the sampler for collecting a blood sample comprises a capillary tube. In some embodiments the buffer module containing a buffer comprises a two-piece cap. In some embodiments the buffer comprises an extraction buffer with a detergent. In some embodiments the kits further comprise an optical reader configured to detect a colorimetric intensity and/or fluorescence intensity produced by the device.

Accordingly, it is an object of the presently disclosed subject matter to provide an inexpensive, easy-to-use, point-of-care test or assay to measure HbS levels in a patient sample. This and other objects are achieved in whole or in part by the presently disclosed subject matter. Further, an object of the presently disclosed subject matter having been stated above, other objects and advantages of the presently disclosed subject matter will become apparent to those skilled in the art after a study of the following description, Drawings and Examples.

### BRIEF DESCRIPTION OF THE DRAWINGS

The presently disclosed subject matter can be better understood by referring to the following figures. The components in the figures are not necessarily to scale, emphasis instead being placed upon illustrating the principles of the presently disclosed subject matter (often schematically). In the figures, like reference numerals designate corresponding parts throughout the different views. A further understanding of the presently disclosed subject matter can be obtained by reference to an embodiment set forth in the illustrations of the accompanying drawings. Although the illustrated embodiment is merely exemplary of systems for carrying out the presently disclosed subject matter, both the organization and method of operation of the presently disclosed subject matter, in general, together with further objectives and advantages thereof, may be more easily understood by reference to the drawings and the following description. The drawings are not intended to limit the scope of this presently disclosed subject matter, which is set forth with particularity in the claims as appended or as subsequently amended, but merely to clarify and exemplify the presently disclosed subject matter.

For a more complete understanding of the presently disclosed subject matter, reference is now made to the following drawings in which:

FIGS. 1A through 1C are schematic illustrations of a lateral-flow immunoassay (LFIA) as disclosed herein;

FIGS. 2A and 2B are schematic illustrations of the appearance of a LFIA as disclosed herein when exposed to varying concentrations of HbS;

FIGS. 3A and 3B are illustrations of exemplary point-of-care LFIA devices as disclosed herein;

FIG. 4 is a schematic illustration of a method of measuring HbS in a sample using an LFIA as disclosed herein;

FIG. 5 is a scatter plot of color intensity of a LFIA over an increasing concentration of HbS in a sample;

FIGS. 6A and 6B graphically compare the estimated percentage of HbS using the disclosed LFIA devices, systems and methods, with actual HbS values, with FIG. 6A being a scatter plot of estimated % HbS compared to actual % HbS, and FIG. 6B being a scatter plot comparing the difference between actual and estimated % HbS against the mean of the actual and estimated % HbS; and

FIG. 7 is a scatter plot of color intensity of an LFIA as disclosed herein over a range of hemoglobin concentrations for samples with varying concentrations of % HbS. Legend: diamonds, 0% HbS; squares, 40% HbS; and circles, 80% HbS.

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

### Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of a composition, dose, sequence identity (e.g., when comparing two or more nucleotide or amino acid sequences), mass, weight, temperature, time, volume, concentration, percentage, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

The term “gene” refers broadly to any segment of DNA associated with a biological function. A gene can comprise sequences including but not limited to a coding sequence, a promoter region, a cis-regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

As is understood in the art, a gene comprises a coding strand and a non-coding strand. As used herein, the terms “coding strand”, “coding sequence” and “sense strand” are used interchangeably, and refer to a nucleic acid sequence that has the same sequence of nucleotides as an mRNA from which the gene product is translated. As is also understood in the art, when the coding strand and/or sense strand is used to refer to a DNA molecule, the coding/sense strand includes thymidine residues instead of the uridine residues found in the corresponding mRNA. Additionally, when used to refer to a DNA molecule, the coding/sense strand can also include additional elements not found in the mRNA including, but not limited to promoters, enhancers, and introns. Similarly, the terms “template strand” and “antisense strand” are used interchangeably and refer to a nucleic acid sequence that is complementary to the coding/sense strand.

Similarly, all genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Also encompassed are any and all nucleotide sequences that encode the disclosed amino acid sequences, including but not limited to those disclosed in the corresponding GENBANK® entries.

As used herein, the terms “antibody” and “antibodies” refer to proteins comprising one or more polypeptides substantially encoded by immunoglobulin genes or fragments of immunoglobulin genes. The presently disclosed subject matter also includes functional equivalents of the antibodies of the presently disclosed subject matter. As used herein, the phrase “functional equivalent” as it refers to an antibody refers to a molecule that has binding characteristics that are comparable to those of a given antibody. In some embodiments, chimerized, humanized, and single chain antibodies, as well as fragments thereof, are considered functional equivalents of the corresponding antibodies upon which they are based.

The term “substantially identical”, as used herein to describe a degree of similarity between nucleotide sequences, peptide sequences and/or amino acid sequences refers to two or more sequences that have in one embodiment at least about least 60%, in another embodiment at least about 70%, in another embodiment at least about 80%, in another embodiment at least about 85%, in another embodiment at least about 90%, in another embodiment at least about 91%, in another embodiment at least about 92%, in another embodiment at least about 93%, in another embodiment at least about 94%, in another embodiment at least about 95%, in another embodiment at least about 96%, in another embodiment at least about 97%, in another embodiment at least about 98%, in another embodiment at least about 99%, in another embodiment about 90% to about 99%, and in another embodiment about 95% to about 99% nucleotide and/or amino acid identity, when compared and aligned for maximum correspondence, as measured using a sequence comparison algorithm or by visual inspection.

As used herein, the terms “detectable moiety”, “detectable label”, “detectable agent”, “label” and/or “marker” refer to any molecule that can be detected by any moiety that can be added to a chemoprobe, antigen, inhibitor, marker, reagent and/or antibody, or a fragment or derivative thereof, that allows for the detection of the chemoprobe, antigen, inhibitor, marker, reagent and/or antibody, fragment, or derivative in vitro and/or in vivo. Representative detectable moieties include, but are not limited to, nanoparticles, chromophores, fluorescent moieties, radioactive labels, affinity probes, enzymes, antigens, groups with specific reactivity, chemiluminescent moieties, and electrochemically detectable moieties, etc. In some embodiments, the antibodies are biotinylated.

In some embodiments, a detectable moiety comprises a fluorophore. Any fluorophore can be employed with the compositions of the presently disclosed subject matter, provided that the conjugation of fluorophore results in a composition that is detectable either in vivo (e.g., after administration to a subject) and/or in vitro, and further does not negatively impact the ability of the chemoprobe, antigen, inhibitor, marker, reagent and/or antibody, or the fragment or derivative thereof, to bind to its epitope. Representative fluorophores include, but are not limited to 7-dimethylaminocoumarin-3-carboxylic acid, dansyl chloride, nitrobenzodiazolamine (NBD), dabsyl chloride, cinnamic acid, fluorescein carboxylic acid, Nile Blue, tetramethylcarboxyrhodamine, tetraethylsulfohodamine, 5-carboxy-X-rhodamine (5-ROX), and 6-carboxy-X-rhodamine (6-ROX). It is understood that these representative fluorophores are exemplary only, and additional fluorophores can also be employed. For example, there the ALEXA FLUOR® dye series includes at least 19 different dyes that are characterized by different emission spectra. These dyes include ALEXA FLUOR® 350, 405, 430, 488, 500, 514, 532, 546, 555, 568, 594, 610, 633, 635, 647, 660, 680, 700, and 750 (available from Invitrogen Corp., Carlsbad, Calif., United States of America), and the choice of which dye to employ can be made by the skilled artisan after consideration of the instant specification based on criteria including, but not limited to the chemical compositions of the specific ALEXA FLUOR®, whether multiple detectable moieties are to be employed and the emission spectra of each, the detection technique to be employed, etc.

In some embodiments, a detectable moiety comprises a cyanine dye. Non-limiting examples of cyanine dyes that can be conjugated to the chemoprobe, antigen, inhibitor, marker, reagent and/or antibody, fragments, and/or derivatives of the presently disclosed subject matter include the succinimide esters Cy5, Cy5.5, and Cy7, supplied by Amersham Biosciences (Piscataway, N.J., United States of America).

In some embodiments, a detectable moiety comprises a near infrared (NIR) dye. NIR dyes, including for example NIR641 and NIR664, can be conjugated to the chemoprobe, antigen, inhibitor, marker, reagent and/or antibody, fragments, and/or derivatives of the presently disclosed subject matter.

In some embodiments, biotinylated chemoprobes, antigens, inhibitors, markers, reagents and/or antibodies are detected using a secondary antibody that comprises an avidin or streptavidin group and is also conjugated to a fluorescent label including, but not limited to Cy3, Cy5, Cy7, and any of the ALEXA FLUOR® series of fluorescent labels available from INVITROGEN™ (Carlsbad, Calif., United States of America). In some embodiments, the chemoprobe, antigen, inhibitor, marker, reagent and/or antibody, fragment, or derivative thereof is directly labeled with a fluorescent label and enzymes, co-factors, peptides, molecules and/or cells that bind to the antibody are separated by fluorescence-activated cell sorting. Additional detection strategies are known to the skilled artisan.

For applications including but not limited to detection applications and imaging applications, the chemoprobes, antigens, inhibitors, markers, reagents and/or antibodies of the presently disclosed subject matter can be labeled with a detectable moiety. The detectable moiety can be any one that is capable of producing, either directly or indirectly, a detectable signal. For example, a detectable moiety can be a radioisotope, such as but not limited to ³H, ¹⁴C, ³²P, ³⁵S, ¹²⁵I, or ¹³¹I; a fluorescent or chemiluminescent compound such as but not limited to fluorescein isothiocyanate, rhodamine, or luciferin; or an enzyme, such as but not limited to alkaline phosphatase, β-galactosidase, or horseradish peroxidase.

Lateral Flow Immunoassay (LFIA) Systems and Devices

The presently disclosed subject matter generally relates to hematology, reagents and methods for samples of human whole blood, packed red cells, dry blood spot or isolated hemoglobins. The presently disclosed subject matter relates to devices, systems and/or methods for screening and/or testing samples from a subject or patient to detect the presence of and/or quantify HbS. By quantifying HbS in a patient sample the health of the patient can be monitored, particularly with respect to SCD. Moreover, for SCD patients there is a need to monitor HbS blood concentrations before, during and/or after treatment in order to appropriately plan treatments, dosages and monitor progress. For example, a point-of-care (POC) test that could be administered prior to and during treatment, as disclosed herein, can ensure that a patient receives the appropriate amount of RBCs per dose of therapeutic, e.g. hydroxyurea.

The subject disclosure comprises methods for using antibodies to specifically detect and quantify HbS concentration with minimal interference from other hemoglobin variants. The outcome of this disclosure can overcome current clinic treatment work flow limitations with superior performance over the current state of the art by using disclosed immunoassay techniques.

Patients who are under chronic transfusion treatment could have an HbS level from 50% to 60% before transfusion, but patients who are under intermittent transfusion treatment/hydroxyurea treatment may have over 80% HbS level due to individual variations. At least one goal of SCD treatment for patients is to dilute HbS to decrease the viscosity and increase the oxygen saturation of blood. SCD patients undergoing treatment may have 100% HbS in total hemoglobin and lower (<9 g/dL) total hemoglobin concentration. The disclosed devices, systems and methods indicate measurement of HbS in clinically relevant ranges is feasible.

In some embodiments the presently disclosed subject matter relates to devices, systems and/or methods to quantitatively measure hemoglobin S in whole blood specimens of patients, including for example patients undergoing or about to undergo SCD therapy. In some embodiments the devices and/or systems disclosed herein can comprise a test that provides a rapid, simple and accurate point-of-care (POC) diagnostic and screening assay for low-resource settings. Such devices and/or systems can also be characterized in some embodiments as an in vitro diagnostic (IVD) test. Also disclosed are reagent combinations and kits for use in such assays.

The presently disclosed quantitative, diagnostic and/or screening assays, devices, systems and methods pertain at least in part to the following aspects and objects (in no particular order):
the use of non-red-brown colored or fluorescent nanoparticles as a marker/indicator, e.g. blue-dyed latex nanoparticles, instead of using red-brown colored colloidal gold particles to improve contrast with the blood sample in order to qualitatively and/or quantitatively detect HbS for measuring HbS and/or monitoring SCD in a patient;
the design and application of a LFIA to qualitatively and/or quantitatively detect HbS in a sample using a detection antibody chemically conjugated to colored or fluorescent nanoparticle;
the use of buffers to decrease non-specific interactions and background signal;
the use of a combination of antibodies as disclosed herein to get appropriate signal enhancement and low background;
conjugation techniques to limit aggregation of the antibody-nanoparticle conjugates and in some embodiments increase sensitivity depending on the antibody density on the surface of the nanoparticles; and
the use of colorimetric nanoparticle technique to allow visualization of a positive/negative test result in a point-of-care device/apparatus.

Thus, in some embodiments the presently disclosed subject matter relates to devices, systems and/or methods for qualitatively and/or quantitatively measuring HbS in a sample or specimen by chemically conjugated Hb-specific monoclonal antibody (mAb) or polyclonal antibody (pAb) to colored or fluorescent nanoparticles. A specific polyclonal antibody or monoclonal antibody, or combination of antibodies, against human HbS is used as the detection antibody. Each detection antibody can be conjugated to colored or fluorescent nanoparticles and dispensed onto the conjugate pad or other sites on the sample fluidic pathway in lateral flow immunoassay. A human hemoglobin-S chain specific polyclonal antibody or monoclonal antibody can be used as the capture antibody. The detection antibody and the capture antibody are either the same antibody or different antibodies. The capture antibody can be printed as the test line on a membrane, such as a nitrocellulose membrane, and can be configured to specifically bind human hemoglobin-S from human whole blood, and in some embodiments can be multiplexed. The resulting colored or fluorescent test-line (T-line) on the membrane, such as a nitrocellulose membrane, can be used to qualitatively and/or quantitatively determine the concentration, percent or other quantity in a sample, such as for example a blood sample.

In some embodiments the presently disclosed subject matter addresses the problems stated above by the use of an immunoassay platform that incorporates polyclonal and/or monoclonal detection antibodies specifically against human hemoglobin chemically conjugated to colored or fluorescent nanoparticles. In some aspects the devices and systems disclosed herein can be configured as a sandwich immunoassay comprising the detection antibody(ies) against Hb and/or HbS with the other polyclonal or monoclonal capture antibody(ies) dispensed or applied in a test-line on a membrane, such as a nitrocellulose membrane. Such configurations can in some aspects provide a dramatically distinguishable color (non-red-brown color, e.g. dark-blue color) or fluorescent qualitative or quantitative result. The presence or absence of a non-red-brown colored or fluorescent test-line can be observed by the naked eye in some embodiments, and/or by measuring/detecting the color or fluorescence intensity at the test-line using a colorimetric/fluorescence detection apparatus. By way of example and not limitation, in such a configuration the color, fluorescence or other detectable quality can be quantified by using a reader detecting the colorimetric intensity or fluorescence intensity.

Another benefit of the disclosed devices, systems, apparatuses and methods is the chemical conjugation approaches that in some embodiments can permanently and irreversibly link the polyclonal or monoclonal detection antibody with the detectable moiety. By way of example and not limitation, such detectable moiety can comprise nanoparticles, including polystyrene nanoparticles, conjugated to non-colloidal gold or fluorescent compounds. Compared to physical adsorption, this permanent chemical conjugation can provide an irreversible link between the detector antibody and the colored or fluorescent nanoparticle. Therefore, this chemical conjugation can provide stable performance and consistent results over time.

Another benefit of the disclosed devices, systems, apparatuses and methods is the design of the LFIA with the chemically conjugated detection antibody-nanoparticle complex dispensed onto the conjugate pad to avoid desorption of the antibody from the nanoparticle. This can in some embodiments improve LFIA stability, prolong LFIA shelf-life, and maintain test performance consistency over time.

Thus, disclosed herein are qualitative and/or quantitative devices, systems and/or methods configured to detect the presence or absence of human HbS or different levels of HbS in human blood samples for monitoring SCD in a subject by the use of colored or fluorescent nanoparticle instead of using colloidal gold particles. As disclosed herein an aspect of these devices, systems and/or methods includes chemical conjugation of colored or fluorescent nanoparticles with polyclonal or monoclonal human Hb-specific and/or HbS-specific detection antibodies to form a permanent, irreversible antibody-nanoparticle complex instead of using a physical adsorption approach to form temporary, reversible antibody-nanoparticle complex.

In some aspects the presently disclosed subject matter comprises the use of a lateral flow immunoassay using a chemically conjugated anti-Hb and/or anti-HbS detection antibody-nanoparticle complex in either simplexed or multiplexed format to simultaneous detect, quantify, and/or differentiate the presence or absence or the absolute and/or relative levels of HbS and other substances including, but not limited to, chemicals, drugs, proteins, nucleotides, etc.

In some aspects the presently disclosed subject matter can comprise the use of a chemically-conjugated Hb and/or HbS detection antibody with nanoparticles in lateral flow immunoassay, coupled with a HbS capture antibody to detect and/or quantify HbS in order to diagnose, monitor and/or proscribe treatments for SCD and/or related conditions, e.g. other hemoglobinopathies including, but not limited to, types of α-thalassemia 1 (trait or disease), α-thalassemia 2 (trait or disease), β-thalassemias (minor, intermediate, or major types), sickle cell disease or sickle cell trait including, HbAS, HbSS, HbSβ⁺-thalassemia, HbSβ⁰-thalassemia, sickle hemoglobin C disease (HbSC), and HbSX, where X could be another globin chain variant that is not HbA (examples: HbSD-Punjab, HbSO-Arab, HbFS or HbSE); various types of hemoglobin C disease and hemoglobin C trait including: HbC disease (HbCC), HbCβ⁰-thalassemia, HbCβ⁺-thalassemia, hemoglobin C trait (HbAC), etc.

In some aspects the presently disclosed devices, systems and assays can be configured to measure and/or detect HbS in a sample from a patient or subject, such as for example a human subject. Samples can in some embodiments refer to whole blood samples, dried blood samples such as a dry blood spot, packed red cell samples, whole blood samples and/or red blood samples from a blood bank, isolated or purified human hemoglobin protein samples, or freshly collected filter paper samples. In some embodiments a sample can be obtained via a finger-stick, heel-stick or venipuncture, dry blood dot, packed red blood cells, isolated mixing of the sample with reagents for conditioning and adding the resulting conditioned sample to the sample well of the LFIA cassette device.

In some embodiments the disclosed devices, systems and methods comprise lateral flow test strips comprising a component of a competition assay, an indirect assay or a sandwich assay. Such assay configurations can further comprise different forms of immunoassays including, but not limited to, lateral flow immunoassay, enzyme immunoassay (EIA), enzyme-linked immunosorbent assay (ELISA), liquid chromatography (LC), radioimmunoassay (RIA), strip-dip test, dot blot, protein array, microfluidics device, flow cytometry, etc. In some embodiments such an immunoassay system can comprise a substrate upon which the capture antibody can be immobilized, wherein the substrate can comprise a chromatography matrix, a bead or a multiwell plate.

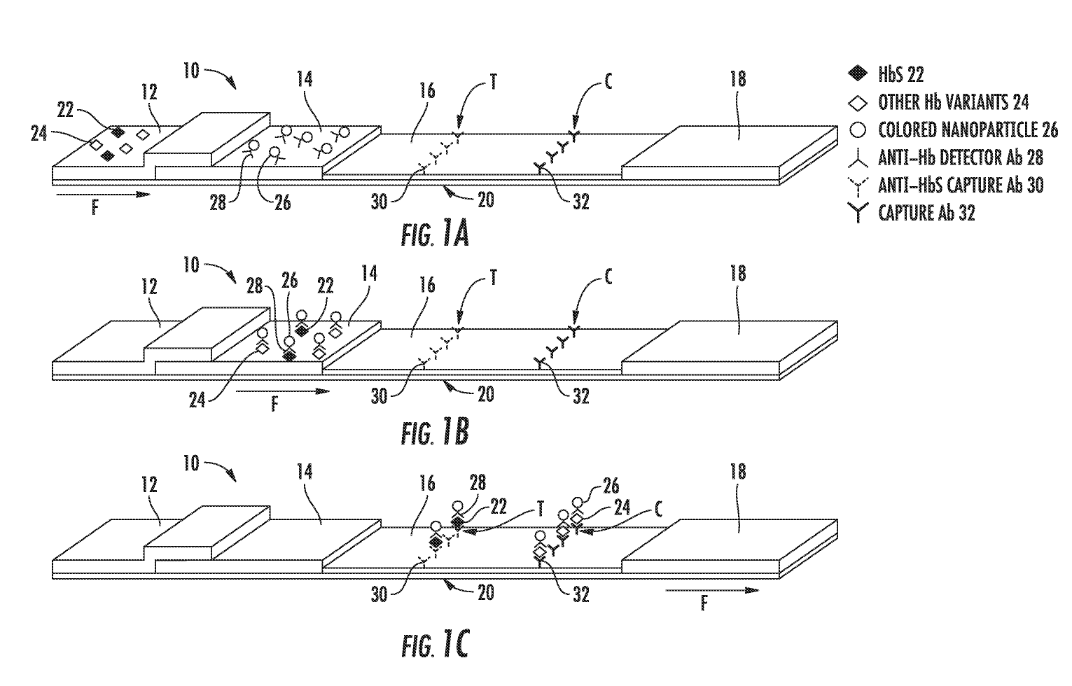

Turning now to the Figures, FIGS. 1A through 10 are schematic illustrations of an exemplary LFIA 10 as disclosed herein. FIGS. 1A through 1C can be viewed in series to illustrate a working principle of at least one embodiment of a LFIA with antibodies for use in quantitative detection of HbS concentration in a sample, such as but not limited to a whole blood sample. As illustrated in FIGS. 1A through 10 a LFIA 10 as disclosed herein can in some embodiments comprise a sample pad 12, conjugate pad 14, a membrane 16, such as a nitrocellulose membrane, and wicking pad 18. These components together can in some aspects be considered a rapid test strip comprising a chromatography matrix, including but not limited to a nitrocellulose membrane, polyvinylidene fluoride membrane, (charge-modified) nylon membrane, polyethersulfone membrane, or the like. That is, one, some or all of the sample pad 12, conjugate pad 14, nitrocellulose membrane 16, and/or wicking pad 18 (or absorbent pad) can comprise a chromatography matrix. LFIA 10 can further comprise a polyclonal or monoclonal detection antibody 28, or anti-Hb detector Ab 28, chemically conjugated to a colored or fluorescent nanoparticle 26, polyclonal or monoclonal capture antibodies, including anti-HbS capture Ab 30 and capture Ab 32, dispensed as the lines test T and control C lines, respectively. Wicking pad 18 can in some embodiments be configured to cause a sample or analyte, including HbS 22 and/or other Hb variants 24 to flow or migrate in a direction F generally from the sample pad 12 to wicking pad 18.

As the test sample diffuses through the absorbent test strip, or LFIA 10, the labeled Hb detector antibody/probe conjugate 28/26 binds to the Hb (analyte; either HbS 22 or other Hb variants 24) in the specimen forming an antibody-antigen complex. The specimen then migrates across a membrane toward the test line T region containing capture HbS antibody T to selectively detect the levels of HbS. The complex of the anti-Hb detector Ab 28 and chemically conjugated colored or fluorescent nanoparticle 26 binds to any HbS in a sample and is captured at the test line T and produces a detectable signal. The probe intensity varies due to different amounts of HbS in each specimen. To serve as a procedural control, a detectable signal always appears at the control line C region if the proper volume of sample has been added and membrane wicking has occurred.

Polyclonal or monoclonal capture antibody 30 can comprise an antibody against human HbS (test T), whereas capture antibody 32 can in some embodiments serve as a control (control C) and can comprise a secondary antibody against hemoglobin (Hb), including for example Hemoglobin alpha chain and/or hemoglobin beta chain, and/or an immunoglobulin of the host animal/subject, including for example IgG. In some embodiments a complex of chemically conjugated Hb-specific detection antibody 28 and colored or fluorescent nanoparticle 26 is sprayed in printing buffer on the conjugate pad 14 and allowed to dry, or any other suitable approach for application/deposition. In some embodiments the concentration of one or more capture antibodies, e.g. anti-HbS capture antibody, can be changed or optimized based on the shape and/or slope of the calibration curve (see Examples below). Thus, a calibration or standard curve can be used to determine an appropriate concentration of capture antibody to be applied to a conjugate pad. In some embodiments the concentration of a capture antibody can range from about 0.1 mg/mL to about 2.0 mg/mL, or about 0.1, 0.5, 0.75, 1.0, 1.25, 1.5, 1.75 or 2.0 mg/mL. A sub-optimally low concentration of capture antibody may produce too low of a signal intensity. A sub-optimally high concentration of the capture antibody may produce a high signal intensity that quickly plateaus without providing sufficient correlation to HbS biomarker concentration. An optimal concentration of anti-HbS capture antibody produces a high sensitivity while maintaining a linear correlation.

In some embodiments an antibody-nanoparticle complex 26/28 can be applied to form an analyte capture zone, or test line T and/or control line C, which can in some embodiments be rectangular in shape, circular in shape, and/or some other shape. In some aspects an analyte capture zone, e.g. test line T and/or control line C, can be arranged or configured in a linear array parallel to and/or substantially distant from sample pad 12. In some aspects the sample pad 12 and/or conjugate pad 14 are located in front of either polyclonal or monoclonal detection antibodies against human HbS at test line T and/or control line C such that the antibody-nanoparticle conjugates can attach to the any Hb and/or HbS in the sample as it flows F toward the test line T and/or control line C.

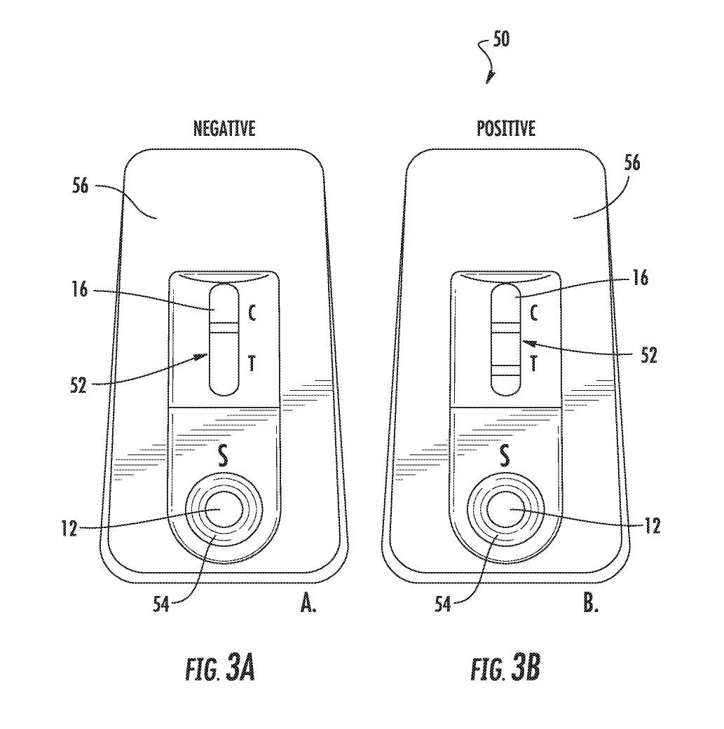

In some embodiments chromatography matrix/nitrocellulose membrane 16 can be overlapped by wicking pad 18 at one end and conjugate pad 14 at the other end. In some embodiments the sample pad 12 can overlap conjugate pad 14, but may not be in contact with nitrocellulose membrane 16. In some embodiments one or more of nitrocellulose membrane 16, conjugate pad 14, sample pad 12, and wicking/absorbent pad 18 can be assembled or held together by a backing material, e.g. a backing card 20, securing each component in a single strip. Such a strip as depicted in FIG. 1 can be assembled and cut to a given width using a precision cutter. The manufactured strip can then be assembled in a disposable cassette (see FIGS. 3A and 3B) and packed under desiccation followed by storage at room temperature until use.

In some embodiments the devices, systems and methods disclosed herein, including for example LFIA device 10 can comprise the use of colored (non-red-brown colored) or fluorescent nanoparticles, instead of using colloid gold particles for detecting and/or quantifying HbS. Because colloidal gold particles have a red-brown color, which is very similar to the color of hemoglobin when running on a lateral flow membrane, it can be difficult to distinguish a red-brown colored positive test-line from the hemoglobin background. This poor visibility can decrease the sensitivity and specificity of a lateral flow immunoassay when determining a concentration of HbS in a sample. Thus, a component of the presently disclosed subject matter is the use of non-red-brown colored (e.g. dark blue colored) nanoparticles. Alternatively, in some embodiments this approach can applied on fluorescent nanoparticles. These fluorescent nanoparticles can, for example, emit pink-red fluorescence or other colors after excited by ultraviolet light or other light. Both approaches can provide significant improvement on the test-line visibility which makes the test-line easily distinguishable from the hemoglobin background.

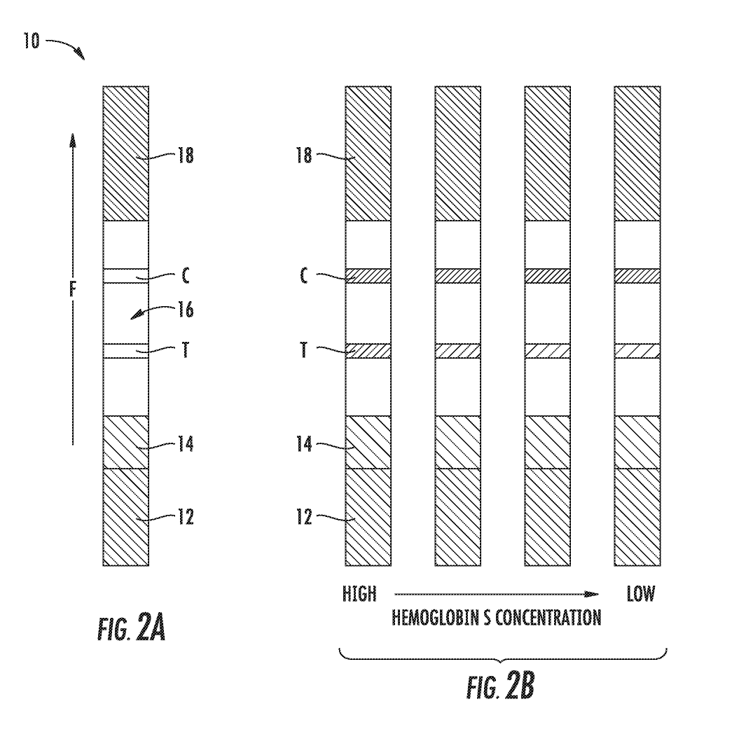

FIGS. 2A and 2B are schematic illustrations of the design of a LFIA, with FIG. 2A illustrating the general orientation of components of the LFIA, and FIG. 2B showing the appearance of four exemplary LFIA when exposed to varying concentrations of HbS (highest HbS concentration on the left and lowest HbS concentration on the right). Corresponding to FIGS. 1A, 1B and 1C, the LFIA in FIG. 2A shows a sample pad 12, conjugate pad 14, nitrocellulose membrane 16, and wicking pad 18. A polyclonal or monoclonal capture antibody can comprise an antibody against human HbS at the test line T, whereas a secondary antibody against hemoglobin (Hb) of the host animal/subject can be present at the control line C. As shown in FIG. 2B, the higher the concentration of HbS in a given sample the greater the intensity of the color of the test line T, the absorbance or fluorescence of which can be observed and/or quantitatively measured. By way of example and not limitation, FIG. 2B shows four LFIA strips exposed to blood samples with varying HbS concentration (highest HbS concentration on the left and lowest HbS concentration on the right). When the HbS concentration is lower, the test line probe intensity is also lower.

In some embodiments polyclonal or monoclonal Hb-specific detection antibodies 28 that are chemically conjugated to a colored or fluorescent nanoparticle 26 can be provided as permanently and irreversibly coupled to the colored or fluorescent nanoparticle 26. By way of example and not limitation, FIG. 2 of PCT International Application Serial No. PCT/US2016/056260 (herein incorporated by reference in its entirety) illustrates a method of producing, by chemical conjugation, such permanently and irreversibly coupled colored or fluorescent nanoparticles with a detection antibody. In some aspects other detectable markers other than colored or fluorescent nanoparticles can be conjugated to the antibodies, including for example small molecule dyes.

In some embodiments the chemical conjugation, if not involving a linker, can include at least two steps as disclosed in PCT International Application Serial No. PCT/US2016/056260. By way of example and not limitation, a first step can comprise the activation of a chemical group, such as for example a carboxyl group, on the surface of a nanoparticle. In some embodiments, a second step can comprise the coupling of the activated chemical group on the nanoparticle with the other chemical group on the antibody, such as for example amino groups.

The chemical conjugation approaches and reaction strategies may or may not include one or more intermediate linker. Also, the chemical groups on the surface of nanoparticle, detection antibody, or the above linker(s) involved in the above chemical conjugation include, but are not limited to, —COOH, —NH₂, —CHO, —SH, etc.

To elaborate, the detection antibody-nanoparticle conjugation may be accomplished through various chemical processes or methods. By way of example and not limitation, such a conjugation strategy can involve the chemical conjugation of a nanoparticle with a —COOH group on the surfaces with the —NH₂ group on the antibody. In some embodiments this method can comprise a carboxyl activation step and an antibody coupling step. In the carboxyl activation step a nanoparticle with a —COOH group can be mixed with a buffer solution containing EDAC (1-ethyl-3-(3-dimethylaminopropyl) carbodimide hydrochloride (mixed, for example, at room temperature for about 10 minutes to about 1 hour) to form nanoparticle-EDAC ester intermediate. In some embodiments the carboxyl activation step can comprise the use of an NHS or sulfo-NHS is often used to increase the efficiency of coupling. Nanoparticle-EDAC ester intermediate can be conjugated to a —NH2 group on the antibody in an antibody coupling step to form to form an antibody-nanoparticle conjugate. The antibody coupling step can in some embodiments comprise combining a nanoparticle-EDAC ester intermediate with an antibody in a buffer solution and mixing the two for a period of time sufficient to form an antibody-nanoparticle conjugate. Such mixing can be from about 30 minutes to overnight from about room temperature to about 4° C. The antibody-nanoparticle conjugate can be collected via centrifugation in some embodiments and then resuspended in an appropriate buffer as needed, e.g. a bead storage buffer.

In some embodiments an antibody-marker complex can be produced by providing a detectable marker and activating the detectable marker by exposing the detectable marker to a compound configured to activate a carboxyl group on the detectable marker, including for example by forming an ester on the detectable marker, and/or by mixing the detectable marker with buffer and exposing the detectable marker to EDAC (1-ethyl-3-(3-dimethylaminopropyl) carbodiimide hydrochloride. Then an antibody can be coupled to the activated detectable marker by exposing the antibody to the activated detectable marker in a buffer solution. In the resulting conjugate the antibody can be chemically conjugated to the detectable marker to form a permanent, irreversible antibody-marker complex. Any other covalent or non-covalent conjugation techniques can be employed without departing from the scope of the instant application.

In some embodiments, the presently disclosed subject matter provides chemical conjugation of a HbS-specific detection antibody with a nanoparticle, or other detectable moiety/marker, to form a permanent, irreversible antibody-nanoparticle complex to detect human HbS instead of using physical adsorption to form a temporary, unstable, and reversible antibody-nanoparticle complex to detect HbS. Therefore, the presently disclosed subject matter encompasses any suitable form or configuration of chemical conjugation strategies using different chemical groups on the surfaces of nanoparticles and antibodies, including but not limited to, a —COOH group on a nanoparticle, a —NH2 group on an antibody, a tosyl group, and maleimide chemistries. Moreover, the presently disclosed subject matter encompasses any suitable chemical conjugation approaches with or without linkers, including but not limited to, sulfo-NHS linkers.

In some embodiments any suitable detectable particle, marker or moiety can be used, including for example, but not limited to, enzyme labels, fluorescent labels, radiolabels, particulate labels, such as colored latex particles, colored plastic particles including, but not limited to polystyrene, also including those utilized in upconverting phosphor technology.

In some embodiments antibodies used in the disclosed devices, systems and methods can comprise any monoclonal or polyclonal antibody having an affinity to human HbS. In some aspects the antibodies, including the capture antibodies, can include class specific polyclonal and/or monoclonal antibodies directed against different original or modified immunogenic peptide sequences from human HbS. In some embodiments a capture antibody having an affinity to HbS can comprise an antibody having an affinity to an amino acid sequence of SEQ ID NO. 1, wherein SEQ ID NO. 1 can comprise a 14 amino acid sequence of the N-terminus of a mutated human hemoglobin β-chain. The capture antibody located at the analyte capture zone (test line) can be a polyclonal or monoclonal antibody specifically against human HbS and/or any protein or peptide containing the sixth position point mutation of human hemoglobin β-globin chain (sickle cell mutation: β6^{Glu→Val}; SEQ ID NO. 1) and/or its flanking amino acid on both sides except for the terminal cysteine (C). In some embodiments the immunogenic peptide sequence for HbS can comprise a peptide sequence of SEQ ID NO. 1: VHLTPVEKSAVTAC, or a sequence substantially identical thereto.

In some embodiments, a capture antibody having an affinity to HbS can comprise an antibody having an affinity to an amino acid sequence of SEQ ID NO. 2, or the full length amino acid sequence of HbS. See Table 1 below.

TABLE 1
Amino-acid sequence of human hemoglobin S (HbS) beta chain (SEQ ID NO. 2):

| | | | | | |
|---|---|---|---|---|---|
| 10 | 20 | 30 | 40 | 50 | 60 |
| MVHLTPVEKS | AVTALWGKVN | VDEVGGEALG | RLLVVYPWTQ | RFFESFGDLS | TPDAVMGNPK |
| 70 | 80 | 90 | 100 | 110 | 120 |
| VKAHGKKVLG | AFSDGLAHLD | NLKGTFATLS | ELHCDKLHVD | PENFRLLGNV | LVCVLAHHFG |
| 130 | 140 | | | | |
| KEFTPPVQAA | YQKVVAGVAN | ALAHKYH | | | |

In some embodiments a capture antibody having an affinity to the alpha chain of hemoglobin (Hb α-chain), e.g. a capture antibody on the control C line, can comprise an antibody having an affinity to an amino acid sequence of SEQ ID NO. 3. The capture antibody located at the analyte capture zone (control line) can be a polyclonal or monoclonal antibody specifically against human Hb α-chain and/or any protein or peptide with a sequence substantially identical thereto. In some embodiments the immunogenic peptide sequence for Hb α-chain can comprise a peptide sequence of SEQ ID NO. 3 in Table 2 below, or a sequence substantially identical thereto.

TABLE 2
Amino-acid sequence of human hemoglobin alpha chain (SEQ ID NO. 3):

| | | | | | |
|---|---|---|---|---|---|
| 10 | 20 | 30 | 40 | 50 | 60 |
| MVLSPADKTN | VKAAWGKVGA | HAGEYGAEAL | ERMFLSFPTT | KTYFPHFDLS | HGSAQVKGHG |
| 70 | 80 | 90 | 100 | 110 | 120 |
| KKVADALTNA | VAHVDDMPNA | LSALSDLHAH | KLRVDPVNFK | LLSHCLLVTL | AAHLPAEFTP |
| 130 | 140 | | | | |
| AVHASLDKFL | ASVSTVLTSK | YR | | | |

In some embodiments a capture antibody having an affinity to the beta chain of hemoglobin (Hb β-chain), e.g. a capture antibody on the control C line, can comprise an antibody having an affinity to an amino acid sequence of SEQ ID NO. 4. The capture antibody located at the analyte capture zone (control line) can be a polyclonal or monoclonal antibody specifically against human Hb β-chain and/or any protein or peptide with a sequence substantially identical thereto. In some embodiments the immunogenic peptide sequence for Hb β-chain can comprise a peptide sequence of SEQ ID NO. 4 in Table 3 below, or a sequence substantially identical thereto.

TABLE 3
Amino-acid sequence of human hemoglobin beta chain (SEQ ID NO. 4):

| | | | | | |
|---|---|---|---|---|---|
| 10 | 20 | 30 | 40 | 50 | 60 |
| MVHLTPEEKS | AVTALWGKVN | VDEVGGEALG | RLLVVYPWTQ | RFFESFGDLS | TPDAVMGNPK |
| 70 | 80 | 90 | 100 | 110 | 120 |
| VKAHGKKVLG | AFSDGLAHLD | NLKGTFATLS | ELHCDKLHVD | PENFRLLGNV | LVCVLAHHFG |
| 130 | 140 | | | | |
| KEFTPPVQAA | YQKVVAGVAN | ALAHKYH | | | |

FIGS. 3A and 3B are illustrations of exemplary LFIA cassettes, or point-of-care LFIA devices as disclosed herein. Such devices can be configured to accurately detect and/or quantify HbS in a sample from a subject. More particularly, POC cassettes 50 can be configured in some embodiments as qualitative and/or quantitative POC and/or rapid screening tests by visualizing the presence or absence of color or fluorescence intensity of the test-line when screening for HbS on the POC cassette device. Additionally, in some embodiments POC cassettes 50 can be configured as quantitative tests by the use of a colorimetric or fluorescent reader to measure and compare the color or fluorescence intensities of the test line.

As discussed above in connection with FIGS. 1A, 1B, 1C and 2, one aspect of the disclosed LFIA devices, systems and methods that is distinct from other chromatography designs or products is the incorporation of chemically conjugated polyclonal or monoclonal detection antibodies with colored or fluorescent nanoparticles to provide permanent irreversible detection antibody-nanoparticle complexes that are dispensed on to or applied to the conjugate pad. This permanently stable conjugation avoids potential desorption or dissociation of detection antibody from nanoparticles. This provides a technical advantage on the stability and performance consistence of the disclosed LFIA devices, systems and methods, including LFIA POC devices configured to detect HbS.

POC cassettes 50 in FIGS. 3A and 3B comprise a housing 56 surrounding a LFIA, such as for example a LFIA 10 as depicted in FIGS. 1A through 10. Housing 56 further comprises a sample receiving area 54 and test window 52. Housing 56 can be configured to surround a LFIA test strip while providing a place to administer a sample from a patient, e.g. a blood sample, at the sample receiving area 54 and a location to observe the results in the LFIA in test window 52. Sample receiving area 54 can comprise an opening in an upper surface of housing 56 configured to receive a sample and/or allow the sample to be applied to sample pad 12 which is accessible through sample receiving area 54. The presence or absence of HbS in the sample can become apparent by visualizing, either by the naked eye and/or by optically measuring, control C and test T lines on chromatography matrix 16 in test window 52.

FIGS. 3A and 3B depict similar LFIA POC cassettes 50, with FIG. 3A illustrating a negative test result for HbS in a sample (only the control C line is visible), and FIG. 3B showing a positive test result for HbS (both the control C and test T lines are visible). In both FIGS. 3A and 3B the visibilities of the colored and/or fluorescent markers, i.e. antibody-nanoparticle conjugates, at the control C and/or test T lines are distinguishable from hemoglobin background. By way of example and not limitation, the antibody-nanoparticle conjugates used in POC cassettes 50 can be anti-human Hb detection antibody chemically conjugated to pink-red fluorescent nanoparticles (visible under UV light) or dark-blue colored nanoparticles, wherein only HbS is captured by the anti-HbS antibody at the test line T.

There is no currently available diagnostic product on the market with a configuration that includes a chemically-conjugated detection antibody-nanoparticle complex coupled with an HbS capture antibody for detecting and/or measuring HbS in a sample. This colored or fluorescent LFIA device is the first point-of-care device that is able to quantitate HbS in a patient sample quickly and accurately so as to allow for real time therapeutic decisions. Moreover, it is sensitive, specific, rapid, low-cost, easy-to-use with stable shelf-life and consistent performance over time.

Methods and Kits for Measuring HbS

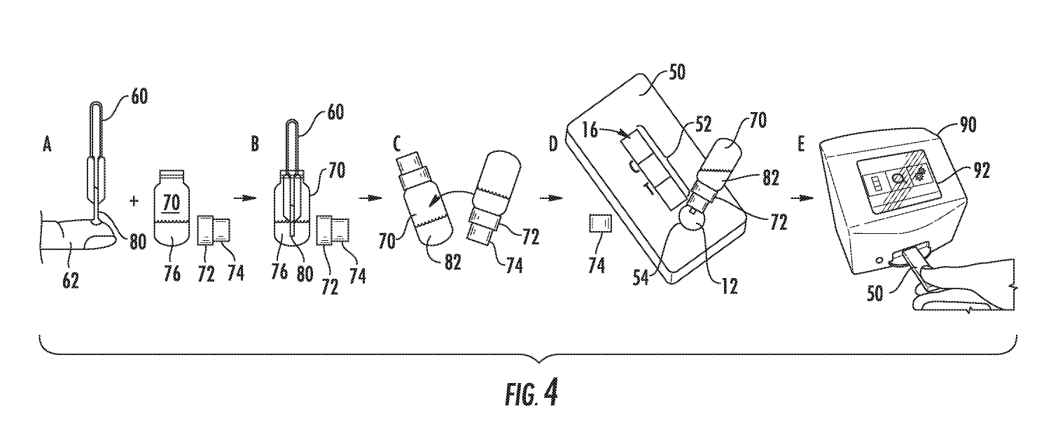

In some embodiments, methods are provided for measuring the concentration of HbS in a sample, e.g. % HbS. In some embodiments, methods are provided for determining the level of circulating HbS in a patient for purposes of assisting in diagnosis, prognosis and treatment design. In some embodiments, such methods, as depicted in FIG. 4 for example, can comprise providing a blood sample (step A), mixing the blood sample with a buffer (steps B and C), providing a LFIA device 50 as disclosed herein, and loading the blood sample mixed in the buffer on the immunoassay device (step D). In some embodiments the LFIA device 50 can be analyzed by an optical reader 90 to quantify the level of HbS in the sample (step E).

In some embodiments an equation or algorithm can be generated to convert test line colorimetric absorbance values to % HbS values based on a number of factors, including but not limited to: test line peak color intensity, test line area color intensity, control line peak color intensity, and control line area color intensity. In some aspects a correlation between intensity (multiple parameters) and concentration can be used to generate % HbS outputs. For example, test line peak color intensity correlating to concentration, test line peak color intensity/control line peak color intensity correlating to concentration, and/or test line area color intensity/control line area color intensity correlating to concentration, can be used. Moreover, in some aspects a lot or batch of cartridges or LFIA tests may have a slightly different algorithm that can be established based on a standard curve to account for variation between batches.

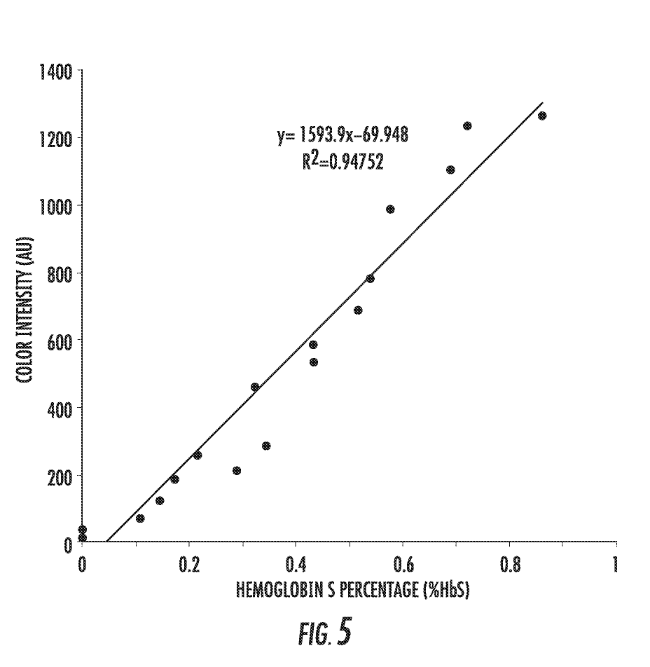

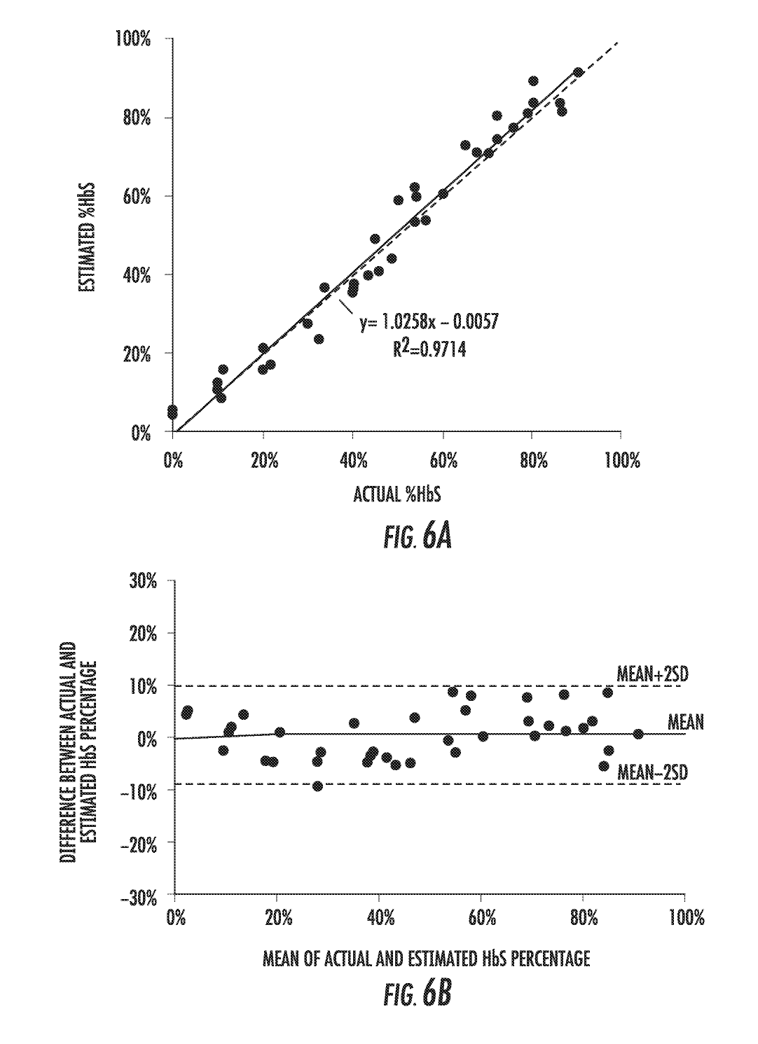

By way of example and not limitation, an equation or algorithm such as that depicted in FIG. 5, or in some embodiments FIG. 6A, can be used to program the reader to generate % HbS results based on a given reading. Such an algorithm or equation can be based on a standard curve as shown in FIG. 5, and can provide an exemplary equation such as y=1593.9x−69.948, where R²=0.94752. Such an algorithm or equation can be based on a plot as shown in FIG. 6A, and can provide an exemplary equation such as y=1.0258x−0.0057, where R²=0.9714.

Such methods are based, at least in part, on the ability to detect the presence of HbS in a sample, and distinguish it from other hemoglobins in the sample. In some embodiments, these methods are based on, and/or provide for, the quantification of the levels of HbS, in absolute or relative terms. Based on the level of HbS in the sample, a medical practitioner can make informed decisions on treatments and therapeutic approaches.

In some embodiments such methods are designed to have a quick turnaround time so as to provide results in a reasonable timeframe for decision making in a filed setting or at the point of care. For example, the runtime for a single measurement can be as little as about 15 minutes, which is sufficiently short for a physician to measure HbS levels during the patient consultation (before transfusion), during the hours long procedure (during the transfusion), and upon completion (after the transfusion). In some embodiments the disclosed LFIA devices and methods can provide HbS concentrations in a little as about 5 minutes to about 1 hour, or about 10 minutes to about 30 minutes, or about 15 minutes or less.

In some embodiments, the methods, as well as LFIA devices, disclosed herein can be based on a blood sample from a subject, e.g. a human subject. In some aspects, the blood sample can be selected from the group consisting of whole blood sample, dried blood sample, packed red cell sample, isolated or purified human hemoglobin protein sample, and freshly collected filter paper sample. A blood sample can be provided using any recognized and suitable approach, including for example finger-stick (step A in FIG. 4), heel-stick and/or venipuncture.

To prepare it for analysis using one of the disclosed methods, devices or systems, a blood sample can be mixed with a buffer comprising a detergent. In some embodiments, the buffer can be optimized to achieve hemolysis, dilution and conditioning of the sample. In some embodiments, the buffer, or extraction buffer, can comprise Brij 30, Tetronic 904, sodium borate and sodium azide, and have a pH of about 8.0. In some embodiments, the buffer can be optimized to lyse/hemolyze the red blood cells contained in the sample, release packed NP-detection antibodies conjugates, create an immunoreaction environment between detection antibodies and hemoglobins, and enhance particle movement on the strip. Once prepared, a sample that has been mixed in such a buffer can be loaded onto a LFIA device, for example, by adding the sample to a sample receiving area of the device.

In some embodiments, kits are provided for the detection and/or quantification of HbS in a sample. FIG. 4 illustrates an exemplary kit and steps for measuring HbS in a sample. As illustrated in FIG. 4, such a kit can comprise a LFIA device 50 or test strip as disclosed herein, a sampler 60 for collecting a blood sample, a buffer module 70 containing a buffer 76, and instructions for performing the HbS measurement. In some aspects, the sampler 60 for collecting a blood sample 80, such as from a subject's finger 62 (step A of FIG. 4), can comprise a capillary tube as illustrated in FIG. 4. The buffer module 70 containing a buffer 76 can in some aspects comprise a two-piece cap having a lower portion 72 and upper portion 74. By removing both the lower portion 72 and upper portion 74 of the cap sampler 60 with the blood sample 80 can be inserted into buffer module 70 such that blood 80 can be added to buffer 76 (step B of FIG. 4).

Continuing with FIG. 4, cap lower portion 72 can be reapplied to buffer module 70 and the contents, blood sample 80 and buffer 76, can be mixed (step C of FIG. 4) to form extract 82. After mixing, upper portion 74 of the cap can be removed such that an appropriate amount (e.g. 5 drops or about 100 ul) of the sample extract 82, or sample and buffer mixture, can be added to sample pad 12 by way of sample receiving area 54 of LFIA 50. Sample extract 82 will migrate or flow through the test strip (as shown in FIGS. 1A, 1B, 1C and 2A) and the results, i.e. intensity of test line T indicating the amount of HbS, will be revealed in window 52. To quantify the HbS shown at test line T on LFIA 50 can be inserted into reader 90 which can be configured to measure the color intensity and/or fluorescence of test line T on LFIA 50 using a colorimeter and/or fluorimeter. Outputs can be displayed on display module 92 of reader 90.

In some embodiments kits are provided herein for screening patients for HbS and/or measuring in a patient relative or absolute levels of HbS. Such kits can comprise for example a POC LFIA device or test as disclosed herein, along with appropriate buffers, solutions, reagents, vials and/or instructions. In some aspects antibody-conjugates can be provided for use in the devices and/or tests. In some aspects a kit can also include a capillary sampler for fingerstick/heelstick blood and a pretreatment module containing buffers for hemolysis of the patient sample. In some embodiments such a kit can further comprise an optical reader as disclosed herein. Such kits can in some embodiments be configured for POC/field use in rapid, one-time use, disposal packages that are cost effective and simple to use.

Subjects

The subject screened, tested, or from which a sample is taken, is desirably a human subject, although it is to be understood that the principles of the disclosed subject matter indicate that the compositions and methods are effective with respect to invertebrate and to all vertebrate species, including mammals, which are intended to be included in the term “subject”.

Moreover, a mammal is understood to include any mammalian species in which screening is desirable, particularly agricultural and domestic mammalian species.

The disclosed devices, systems and methods are particularly useful in the testing, screening and/or treatment of warm-blooded vertebrates. Thus, the presently disclosed subject matter concerns mammals and birds.

More particularly, provided herein is the testing, screening and/or treatment of mammals such as humans, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, i.e., poultry, such as turkeys, chickens, ducks, geese, guinea fowl, and the like, as they are also of economic importance to humans. Thus, provided herein is the treatment of livestock, including, but not limited to, domesticated swine (pigs and hogs), ruminants, horses, poultry, and the like.

In some embodiments, the subject to be used in accordance with the presently disclosed subject matter is a subject in need of treatment and/or diagnosis. In some embodiments, a subject can have or be believed to a hemoglobinapothy or related condition, such as for example sickle cell disease. In some embodiments, a subject can be in need of a therapy that is predicated on determining a level of HbS in the subject, such as for example a transfusion or hydroxyurea treatment for sickle cell disease.

### EXAMPLES

The following examples are included to further illustrate various embodiments of the presently disclosed subject matter. However, those of ordinary skill in the art should, in light of the present disclosure, appreciate that many changes can be made in the specific embodiments which are disclosed and still obtain a like or similar result without departing from the spirit and scope of the presently disclosed subject matter.

### Example 1

### Experimental Design

Blood Samples

The collection and use of all samples was approved by the institutional review board (IRB) of the Medical University of South Carolina. Patients were recruited from regular SCD clinical populations. After receiving written informed consent from patients, venipuncture whole blood samples were collected in K₂EDTA vacutainer tubes (Becton Dickinson, United States of America). Samples were analyzed on-site using HPLC (Variant II Turbo HPLC system, Bio-Rad, United States of America) and the results were provided with hemoglobin concentrations and treatment history. Blood samples were sent to the BioMedomics research facility (BioMedomics, Inc., Durham, N.C., United States of America) for testing using the disclosed HbS tests. Any samples, devices, or components that potentially had contact with blood were disposed of as infectious medical waste according to federal, state, and local requirements.

LFIA Tests

An LFIA test strip is comprised of a series of materials as disclosed herein, including for example sample pad (GE Healthcare, United States of America), glass fiber conjugate pad (GE Healthcare), nitrocellulose membrane (EMD Millipore, United States of America), and absorbent pad (Ahlstrom, United States of America), which were cut and placed into a plastic cartridge. The detector, anti-Hb (0.04 mg/mL, mouse monoclonal antibody, Fitzgerald Industrial International, United States of America) conjugated blue-dyed nanoparticle (2 mg/mL, Bangs Laboratories, Inc., United States of America), was prepared by standard EDC chemistries and applied to the glass fiber conjugate pad. The finial concentration of blue-dyed nanoparticles was evaluated using Tecan (Switzerland). The nitrocellulose membrane was printed as capture region with anti-HbS (0.5 mg/mL, rabbit monoclonal antibody, BioMedomics) or anti-Hb antibody (1 mg/mL, mouse monoclonal antibody, Fitzgerald Industrial International, United States of America) by using IsoFlow system (Imagene, United States of America). The detector particle, hemoglobin, and the capture antibody immobilized on the nitrocellulose form an immune-sandwich complex as the sample flows from the conjugate pad across the nitrocellulose membrane and into the absorbent pad. None of materials are pretreated. Visible lines at the capture regions form in the presence of the specific hemoglobin due to the presence of dyed nanoparticle. 4 mm strips is cut and placed in lateral flow cartridges with exposed window for test strip reading and application site for sample dispensing.

LFIA Testing and Reading Procedure

Testing using an LFIA as disclosed herein occurred within 0-14 days after venipuncture collection from the SCD patient. Immediately before testing, a small volume of the whole blood sample (5 μL) was diluted in 1 mL of PreTreatment Buffer (BioMedomics), causing cell lysis and allowing access to hemoglobin. The buffer treated sample was immediately placed into the LFIA cartridge for detection and quantification of HbS. After allowing 15 minutes of run time, the cartridge was placed into a small, desktop quantitative reader (BioMedomics) for image analysis. The reader illuminated the cartridge with a red LED, and imaged the test and control capture regions of the test strip. The reader software identified the presence of blue lines on the test strip and quantified colorimetric absorbance.

HbS blood samples (from AA patients and SS patients; normal (AA) and sickle cell disease (SS)) were mixed to create desired levels within the range of 0-86% HbS. These varying level blood samples were used to create a calibration curve, which was used to convert test line colorimetric absorbance values to % HbS values traditionally used in the field. The calibration curve was programmed into the reader to direct % HbS result output to the user. By way of example and not limitation, an equation or algorithm such as that depicted in FIG. 5, or in some embodiments FIG. 6A, can be used to program the reader to generate % HbS results based on a given reading. Such an algorithm or equation can be based on a standard curve as shown in FIG. 5, and can provide an exemplary equation such as y=1593.9x−69.948, where R²=0.94752. Such an algorithm or equation can be based on a plot as shown in FIG. 6A, and can provide an exemplary equation such as y=1.0258x−0.0057, where R²=0.9714.

The test was run by dispensing 100 μL of diluted blood sample onto the cartridges sample well and then inserted into the reader after a fixed waiting time, e.g. about 15 minutes. The colorimetric absorbance of the test line was analyzed using the quantitative reader. The test line absorbance on the strip was illuminated with a red LED and quantified by the reader. The output was a quantitative value based on the average colorimetric absorbance of pixels within a specified area on the test. A calibration curve (FIG. 5) was created by converting test line colorimetric absorbance values to known HbS levels. The reader analyzed absorbance levels and output Hb levels in percentages for the end user.

FIG. 5 shows spiked whole blood samples result in test-lines with increasing colorimetric densities. The test-lines were measured and used to generate concentration curves. The calibration curve data was used to program a test-specific algorithm into a small footprint reader to measure and report HbS levels.

Method Comparison to Central Laboratory

A linear regression was generated by correlating the calculated % HbS using a quantitative reader algorithm with clinical HPLC data for % HbS (n=38). A Bland-Altman plot was constructed to compare the difference between the two methods of obtaining % HbS.

Sensitivity and Specificity

A sensitivity study was performed by mixing HbA and HbS blood standards at different ratios to create low HbS level standard samples in the range of 1%-10% HbS. Limit of Blank (LoB) of LFIA tests was estimated by measuring replicates (n=5) of test strips with only buffer, calculating the mean HbS levels of blank samples and the standard deviation (SD) of blank samples, and following the equation of LoB=mean (blank)+1.645×SD (blank). Limit of Detection (LoD) of LFIA tests was determined by measuring replicates (n=5) of blood samples with 2% HbS and calculating according to LoD=LoB+1.645×SD (2% HbS blood sample). Limit of Quantitation (LoQ) of LFIA tests was determined by measuring replicates (n=5) of blood samples with low HbS levels and choosing the lowest level that results in a coefficient of variation (CV) 20%. AA standard (100% HbA, Sigma, United States of America), AF standard (64% HbA and 34.9% HbF, ACS, United States of America), AC standard (62% HbA and 38% HbC, ACS), AD standard (63% HbA, 35.4% HbD and 1.6% HbA2, ACS) and AE standard (68% HbA and 32% HbE, ACS) were used for specificity study.

Interference Testing

Interfering factors commonly found in SCD patient samples can cause false (positive or negative) results. As such, high concentrations of protein (serum albumin), bilirubin, cholesterol, penicillin, and hydroxyurea (all materials available from Sigma Aldrich) were spiked into normal (AA) and sickle cell disease (SS) samples with known HbS levels.

Hemoglobin Concentration Variation

The concentrations of HbA and HbS blood standards (ferrous stabilized human, Sigma) were used to prepare samples with hemoglobin concentration ranging from 0 g/dL to 15 g/dL with varying dilution ratios.

### Example 2

### Design of Workflow and LFIA Devices and Systems

Workflow of the LFIA

SCD patients rely on blood transfusions provided by sickle cell centers. Physicians treating patients rely on hemoglobin quantification by central laboratory HPLC or electrophoresis tests to make clinical decisions about the frequency of transfusions and quantity of blood units transfused. For example, a SCD patient with over 80% HbS concentration will be transfused with normal HbA blood to target a technical process (requiring treatment of sample, maintenance, data analysis, and a significant level of training) that requires bulky, expensive equipment, turnaround time for hemoglobin quantification for SCD patients can be several days to weeks. Without immediate results of hemoglobin levels, physicians treating SCD patients rely on outdated and potentially inaccurate results to guide treatment.

Thus, developed and disclosed herein is a POC test to quantify % HbS near the SCD patient during transfusion therapy. The disclosed devices, systems and methods utilize a LFIA platform with antibodies for HbS and a small-footprint reader (dimensions 12 cm×11.5 cm×11 cm, weight 550g). The runtime for a single measurement cab be as little as about 15 minutes, which is sufficiently short for a physician to measure HbS levels during the patient consultation (before transfusion), during the hours long procedure (during the transfusion), and upon completion (after the transfusion).

Components of the LFIA

The HbS tests, systems, kits and devices disclosed herein can in some embodiments comprise a set of disposable, one-time use materials, including for example a LFIA cartridge, module with pretreatment buffer, and 5 μL capillary sampler, as well as a small-footprint reader (see FIG. 4 for example). Cartridges can be packaged in sealed aluminum foil laminated polyester pouches to protect against exposure to sunlight. The pouches can also contain a desiccant packet to maintain a less than 10% relative humidity of the cartridge package.

A patient sample can be transferred by a capillary sampler, to a module to be mixed with pre-treatment buffer, which in some embodiments can lyse blood cells to allow access to the hemoglobins. The sample may be drawn by finger stick or from a venipuncture sample, which may be depend on consideration of what may be less invasive to the patient. While finger stick is often considered to be less invasive, healthcare providers have access to the vein of these patients already receiving transfusion therap.

A 100 uL aliquot of the treated sample was dropped into the two-piece cartridge, which holds the test strip. Each test strip comprises a series of laminated porous materials that have been sprayed or printed with reagent and dried (FIGS. 1A, 1B, 1C and 2). Within the cartridge, the treated blood sample encounters detector particles coated with anti-hemoglobin, and continues to flow toward the capture region. The capture region is coated with antibodies specific for hemoglobin S, and completes the formation of a sandwich immune-complex with the coated detector particle in the presence of hemoglobin S (FIGS. 1A, 1B, 1C and 2).

Development of Antibodies

Rabbit anti-HbS polyclonal antibodies (pAbs) were developed for the β chain mutation sites in HbS. The polyclonal antisera was evaluated for performance using a standard ELISA method. The resulting antisera were pooled and affinity purified and non-specific Ab depleted. Rabbit antibodies have shown an improved immune response to small epitopes and better recognition of a more diverse range of epitopes compared to mouse antibodies, with other useful qualities to the rabbit's immune system which is able to generate a larger range of high affinity antibodies.

In order to improve the consistency of the test and specificity, monoclonal antibodies (mAbs) against HbS were produced. Using splenocytes from a rabbit that successfully produced pAbs, mAbs were prepared and compared to pAbs using ELISA and LFIA. Significant batch-to-batch variation of pAbs were observed due to varying immune response between animals and immunization periods (i.e. 1^{st} vs 2^{nd} immunization). The monoclonal antibodies, being produced from a cultured hybridoma, had reduced variation between batches. By screening splenocytes, a hybridoma with greater specificity for HbS (compared to HbA) was also created.

Development of the Concentration Curve

A concentration curve was created using three normal blood samples with phenotype HbAA (all 0% HbS) and three SCD blood samples with phenotype HbSS (86.1% HbS, 53.9% HbS, and 72.1% HbS). The samples were mixed to create three sets of samples ranging from 0% HbS to 86% HbS. The % HbS of the SCD blood samples were determined by HPLC and used to calculate the final % HbS of the samples after mixing with normal blood samples. From these three distinct mixtures a summative (n=18) concentration curve was plotted (n=3 for each data point) using a linear regression [% HbS=(colorimetric absorbance+69.948)/1593.9] with agreement R²=0.95 to ensure more accurate determinations of % HbS.

### Example 3

### LFIA Performance Evaluation

Accuracy

FIGS. 6A and 6B illustrate the correlation of the LFIA HbS test to the central laboratory HPLC method. All 38 blood samples were tested by HPLC before use. Five of the samples were used for calibration. The whole blood samples (n=14; 2 AA, 1 AS, 2 SC, 9 SS—normal (AA), AS genotype (AS), SC genotype (SC), and sickle cell disease (SS)) and artificial samples (n=24) mixed by whole blood ultimately yielded data points (n=38, in the range of 0-90% HbS) within close proximity to the curve (slope=1.0258), with high linear agreement between the clinically collected HPLC data and the data presented by the LFIA (FIG. 6A, dashed line). The data points were also highly correlated (R²=0.97) with a linear fit [% HbS from prototype=1.0258×% HbS from HPLC−0.0057]. In the constructed Bland-Altman plot seen in FIG. 6B, the two methodologies showed strong agreement, with a standard deviation of 4.6% HbS. The limits of agreement between the LFIA tests and the clinically collected data were −8.7% HbS and 9.9% HbS. All the LFIA data fell within ±10% HbS difference between LFIA and HPLC data. Among the 38 samples, 9 whole blood samples and 13 artificial samples mixed by whole blood have % HbS in the pre-transfusion level (>40% HbS), and two whole blood samples and 8 artificial samples have % HbS in the post-transfusion level (20-40%). The mean of difference (the SD of difference) of the measurement between LFIA and HPLC is 4.06% (SD=2.89%) for samples in the pre-transfusion level and 3.95% (SD=2.12%) for samples in the post-transfusion level. Currently physicians are using a formula with initial % HbS, body weight and Hb concentration to estimate the post-transfusion level. Piety et al. (2015) did five case studies and showed that the clinical estimation has a 3.3% HbS mean difference in comparison with Hb electrophoresis. The instant results show the LFIA test has the ability to accurately measure % HbS as clinical estimation.

Precision

Experiment to evaluate the precision of the assay was accomplished by measuring repeating (n=20) the % HbS for blood samples obtained from the AA, AS and SS subjects according to the guideline, EP15-A2 “User Verification of Performance for Precision and Trueness” by the Clinical and Laboratory Standards Institute (CLSI). The standard deviation (SD) for the % HbS measurements performed with the same blood sample under the same operational conditions was consistently less than 10% HbS for all values of the HbS level tested: 2% HbS for AA, 5% HbS for AS; and 10% HbS for SS. The coefficients of variation (% CV) were 20% CV for AA, 6% CV for AS, and 9% for SS. Notably, the relatively high % CV for the AA phenotype was due to the low level of % HbS (the biomarker measured).

Sensitivity

The HbS quantitative test was determined to have a 1% HbS limit of blank and 2% HbS limit of detection. The limit of quantitation is 5% HbS, the lowest concentration at which the readings can be considered reliable.

Specificity

AA, AF, AC, AD and AE standard all showed as <2% HbS and therefore no binding with HbS test line. Twelve whole blood samples used in accuracy study (i.e. 2 AA, 1 AS, 2 SC and 7 SS including 5.9-24% HbF) were also evaluated. The mean of difference (the SD of difference) of the measurement between LFIA and HPLC was 4.79% (SD=0.43%) with samples including HbA, 2.37% (SD=2.25%) with samples including HbC and 4.51% (SD=3.27%) with samples including HbF.

Interference Testing High concentrations of total protein, bilirubin, cholesterol, penicillin and hydroxyurea present in a patient blood sample could potentially interfere with the accuracy of results. These interfering factors are often found in SCD patients and have the potential to change the antibody-antigen (hemoglobin) affinity and cause false results. These potential interfering factors in normal (AA) and SCD (SS) samples with known HbS levels were spiked to test the effect on test. Results demonstrate 0% interference with the potentially interfering substances at the following concentrations: protein (serum albumin), 100 mg/mL; bilirubin, 20 mg/dL; cholesterol, 5 mg/mL; hydroxyurea, 75 pg/mL; and penicillin, 500 pg/mL.

Hemoglobin Concentration Effect

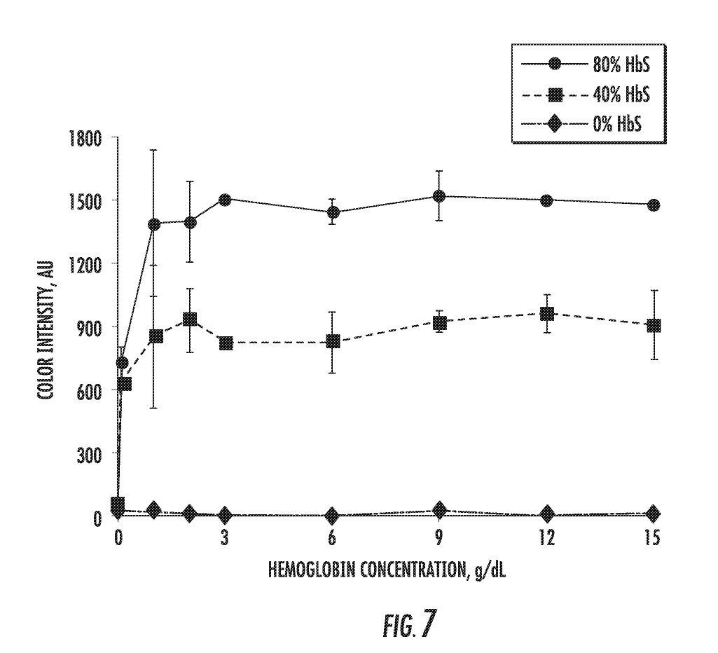

Typical measurements of hemoglobin concentrations are expressed in g/dL, but convention in the field expresses hemoglobin variants, such as HbS, in percent of the total hemoglobin concentration. The effect of variation of the hemoglobin concentration of blood samples was determined in the disclosed HbS tests, methods and systems. Three hemoglobin standard samples of varying total hemoglobin concentrations from 0 to 15 g/dL with HbS concentrations of 0%, 40% and 80% HbS were prepared and tested. FIG. 7 shows the results of this experiment when measured for colorimetric absorbance by the cartridge reader. Within the range of 3 to 15 g/dL total hemoglobin, the colorimetric absorbance of the different % HbS levels are in plateau.

The results indicate that the disclosed devices, systems and methods will accurately measure HbS concentration in a patient sample regardless of total hemoglobin concentration variation. SCD patients have highly variable total hemoglobin concentration, and this concentration can further fluctuate with application of units of blood transfused. Adding to complexity are two transfusion methodologies: (1) simple, in which RBCs are added, and (2) exchange, in which RBCs are removed and replaced with healthy RBCs. In addition, healthy individuals may have a range of hemoglobin concentration between 12 to 15 g/dL.

### Example 4

Summary of Results

For the purposes of monitoring transfusion therapies for sickle cell patients, a point-of-care quantitative immunoassay for HbS was developed and disclosed herein. In some embodiments the platform developed comprises a small-footprint reader and consumable one-time-use cartridge that can quantify the percentage of HbS in a small volume of patient blood.

Using an algorithm developed by a calibration curve, the test reports % HbS values that correlate closely (slope 1.03, R2=0.97) with a currently used central laboratory HPLC system. Coefficients of variation (% CV) for prepared AA, AS and SS samples containing 0%, 40%, and 87% of HbS were 20% CV, 6% CV, and 9% CV, respectively. The test was also shown to encounter minimal interference from high concentrations of protein, bilirubin, cholesterol, hydroxyurea, and penicillin. In addition, within the (total) hemoglobin concentration range of 3 to 15 g/dL, % HbS values were not affected by varying concentrations of total hemoglobin.

### REFERENCES

All references listed herein including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.
1. Powars D, Wilson B, Imbus C, Pegelow C, Allen J. The natural history of stroke in sickle cell disease. Am J Med. 1978; 65(3):461-71.
2. Scothorn D J, Price C, Schwartz D, Terrill C, Buchanan G R, Shurney W, et al. Risk of recurrent stroke in children with sickle cell disease receiving blood transfusion therapy for at least five years after initial stroke. J Pediatr. 2002; 140(3):348-54. doi: 10.1067/mpd.2002.122498.
3. DeBaun M R, Gordon M, McKinstry R C, Noetzel M J, White D A, Sarnaik S A, et al. Controlled trial of transfusions for silent cerebral infarcts in sickle cell anemia. The New England journal of medicine. 2014; 371(8):699-710.
4. Danielson C F. The role of red blood cell exchange transfusion in the treatment and prevention of complications of sickle cell disease. Ther Apher. 2002; 6(1):24-31.
5. Charache S, Terrin M L, Moore R D, Dover G J, Barton F B, Eckert S V, et al. Effect of hydroxyurea on the frequency of painful crises in sickle cell anemia. Investigators of the Multicenter Study of Hydroxyurea in Sickle Cell Anemia. The New England journal of medicine. 1995; 332(20):1317-22
6. Steinberg M H, Barton F, Castro O, Pegelow C H, Ballas S K, Kutlar A, et al. Effect of hydroxyurea on mortality and morbidity in adult sickle cell anemia: risks and benefits up to 9 years of treatment. JAMA. 2003; 289(13):1645-51.
7. Nagel R L, Bookchin R M, Johnson J, Labie D, Wajcman H, Isaac-Sodeye W A, et al. Structural bases of the inhibitory effects of hemoglobin F and hemoglobin A2 on the polymerization of hemoglobin S. Proc Natl Acad Sci USA. 1979; 76(2):670-2.
8. Hankins J, Aygun B. Pharmacotherapy in sickle cell disease—state of the art and future prospects. British journal of haematology. 2009; 145(3):296-308.
9. Ware R E, Helms R W, Investigators S W. Stroke With Transfusions Changing to Hydroxyurea (SWiTCH). Blood. 2012; 119(17):3925-32.
10. Platt O S. Hydroxyurea for the treatment of sickle cell anemia. The New England journal of medicine. 2008; 358(13):1362-9.
11. Nottage K A, Hankins J S, Smeltzer M, Mzayek F, Wang W C, Aygun B, et al. Hydroxyurea use and hospitalization trends in a comprehensive pediatric sickle cell program. PLoS One. 2013; 8(8):e72077.
12. Yawn B P, Buchanan G R, Afenyi-Annan A N, Ballas S K, Hassell K L, James A H, et al. Management of sickle cell disease: summary of the 2014 evidence-based report by expert panel members. JAMA. 2014; 312(10):1033-48.
13. Modell, B.; Darlison, M. Bulletin of the World Health Organization 2008, 86, 480-487.
14. Stuart, M. J.; Nagel, R. L. Lancet 2004, 364, 1343-1360.
15. Trompeter, S.; Roberts, I. British journal of haematology 2009, 144, 308-316.
16. Wanko, S. O.; Telen, M. J. Hematology/oncology clinics of North America 2005, 19, 803-826, v-vi.
17. Chou, S. T. Hematology/the Education Program of the American Society of Hematology. American Society of Hematology. Education Program 2013, 2013, 439-446.
18. Wood, D. K.; Soriano, A.; Mahadevan, L.; Higgins, J. M.; Bhatia, S. N. Science translational medicine 2012, 4, 123ra126.
19. Bartolucci, P.; Brugnara, C.; Teixeira-Pinto, A.; Pissard, S.; Moradkhani, K.; Jouault, H.; Galacteros, F. Blood 2012, 120, 3136-3141.
20. Piety, N. Z.; Yang, X.; Lezzar, D.; George, A.; Shevkoplyas, S. S. American journal of hematology 2015, 90, 478-482.
21. Yetisen, A. K.; Akram, M. S.; Lowe, C. R. Lab on a Chip 2013, 13, 2210-2251.
22. Bland, J. M.; Altman, D. G. Lancet 1986, 1, 307-310.
23. Rossi, S.; Laurino, L.; Furlanetto, A.; Chinellato, S.; Orvieto, E.; Canal, F.; Facchetti, F.; Dei Tos, A. P. American journal of clinical pathology 2005, 124, 295-302.

It will be understood that various details of the presently disclosed subject matter may be changed without departing from the scope of the presently disclosed subject matter. Furthermore, the foregoing description is for the purpose of illustration only, and not for the purpose of limitation.

1. A hemoglobin S (HbS) diagnostic or screening apparatus comprising:
a chromatography matrix;
a detection antibody with binding affinity to hemoglobin (Hb);
a detectable marker conjugated to the detection antibody; and
one or more capture antibodies, wherein the capture antibodies have a binding affinity to HbS, Hb α chain, and/or Hb β chain;
wherein the diagnostic or screening apparatus is configured as a lateral-flow immunoassay, and
wherein the diagnostic or screening apparatus is configured to detect HbS in a sample from a subject.

2. The diagnostic or screening apparatus of claim 1, wherein the detection and/or capture antibodies are monoclonal antibody (mAb) and/or polyclonal antibody (pAb).

3. The diagnostic or screening apparatus of claim 1, wherein the detectable marker comprises a colored or fluorescent nanoparticle.

4. The diagnostic or screening apparatus of claim 1, wherein the diagnostic or screening apparatus is configured to qualitatively and/or quantitatively detect and/or measure human HbS in human blood samples.

5. The diagnostic or screening apparatus of claim 1, wherein the detection antibody is chemically conjugated to the detectable marker to form a permanent, irreversible antibody-marker complex.

6. The diagnostic or screening apparatus of claim 1, wherein the sample from a subject is selected from the group consisting of a whole blood sample, a dried blood sample, a packed red cell sample, a whole blood sample or red blood sample from a blood bank, an isolated or purified human hemoglobin protein sample, and a freshly collected filter paper sample.

7. The diagnostic or screening apparatus of claim 1, wherein the detectable marker is an enzyme label, fluorescent label, radiolabel, particulate label, colored latex particle, colored plastic particle, and a phosphor particle.

8. The diagnostic or screening apparatus of claim 1, wherein at least one of the capture antibodies has a binding affinity to HbS, optionally wherein the capture antibody comprises an antibody having an affinity to an amino acid sequence of SEQ ID NO. 1, wherein SEQ ID NO. 1 comprises an immunogenic amino acid sequence of HbS.

9. A point-of-care (POC) immuno assay device comprising:
a chromatography maxtrix;
a sample pad;
a conjugate pad;
a detection antibody with binding affinity to a hemoglobin (Hb);
a detectable marker conjugated to the detection antibody;
one or more capture antibodies, wherein the capture antibodies have a binding affinity to hemoglobin S (HbS), Hb α chain, and/or Hb β chain; and
a wicking pad,
wherein the device is configured to detect and/or quantify HbS in a sample from a subject at the point-of-care or in a field setting.

10. The device of claim 9, wherein the POC immuno assay is configured as a lateral-flow immunoassay.

11. The device of claim 9, wherein the immuno assay device is configured to assist in monitoring or guiding the transfusion therapy of sickle cell disease (SCD) patients.

12. The device of claim 9, further comprising a housing configured to substantially enclose the chromatography maxtrix, sample pad, conjugate pad, detection antibody, capture antibodies and wicking pad, optionally in a disposable one-time use package.

13. The device of claim 12, further comprising a sample receiving area configured to receive a sample and a test window configured to allow observation of test results.

14. The device of claim 9, wherein the wicking pad is configured to cause a sample to migrate in a lateral direction from the sample pad to the wicking pad by capillary action.

15. The device of claim 9, wherein the one or more capture antibodies can be applied to the chromatography matrix and configured as a test line and control line, wherein both the test line and control line are visible in the test window.

16. The device of claim 9, wherein the detection and/or capture antibodies are monoclonal antibody (mAb) and/or polyclonal antibody (pAb).

17. The device of claim 9, wherein the capture antibodies further comprise an anti-HbS capture antibody and an anti-Hb capture antibody, wherein the anti-HbS capture antibody has an affinity to HbS, wherein the anti-Hb capture antibody has an affinity to Hb α chain and/or Hb β chain.

18. The device of claim 9, wherein the detectable marker comprises a colored or fluorescent nanoparticle.

19. The device of claim 9, wherein the detection antibody is chemically conjugated to the detectable marker to form a permanent, irreversible antibody-marker complex.

20. The device of claim 9, wherein the detectable marker is an enzyme label, fluorescent label, radiolabel, particulate label, colored latex particle, colored plastic particle, and a phosphor particle.

21. The device of claim 9, wherein the detection antibody conjugated to the detectable marker is provided in a simplexed or multiplexed format to simultaneous detect, quantify and/or differentiate the presence or absence of HbS in a sample.

22. A method for quantifying hemoglobin S (HbS) in a patient sample, comprising:
providing a sample from a patient;
providing the diagnostic or screening apparatus of claim 1;
administering the sample to the diagnostic or screening test; and
measuring an intensity of the detectable marker to quantify the HbS in the sample.

23. The method of claim 22, wherein method is configured to provide a quantitative measure of HbS in the sample within about 30 minutes or less.

24. The method of claim 22, wherein the method is configured to assist in monitoring or guiding the transfusion therapy of sickle cell disease (SCD) patients.

25. The method of claim 22, further comprising using an optical reader configured to detect a colorimetric intensity and/or fluorescence intensity.

26. A kit for measuring hemoglobin S in a sample, comprising:
a device of claim 1;
a sampler for collecting a blood sample;
a buffer module containing a buffer; and
instructions for performing measurement of hemoglobin S in the sample.

27. The kit of claim 26, wherein the sampler for collecting a blood sample comprises a capillary tube.

28. The kit of claim 26, wherein the buffer module containing a buffer comprises a two-piece cap.

29. The kit of claim 26, wherein the buffer comprises an extraction buffer with a detergent.

30. The kit of claim 26, further comprising an optical reader configured to detect a colorimetric intensity and/or fluorescence intensity produced by the device.
